(19) United States
(12) Patent Application Publication
(10) Pub. No.: US 2026/0132400 A1
(43) Pub. Date: May 14, 2026

(54) STABILIZED RNA AGENTS

(71) Applicant: A2TBio LLC, Westborough, MA (US)

(72) Inventors: Alexey WOLFSON, Westborough, MA (US); Timofei ZATSEPIN, Westborough, MA (US)

(21) Appl. No.: 18/706,279

(22) PCT Filed: Nov. 1, 2022
(86) PCT No.: PCT/US2022/048509

§ 371 Date: Apr. 30, 2024

Related U.S. Application Data
(60) Provisional application No. 63/274,193, filed on Nov. 1, 2021.

(51) Int. Cl. C12N 15/113; A61K 31/712; A61K 31/7125

(52) U.S. Cl. CPC C12N 15/113; A61K 31/712; A61K 31/7125; C12N 2310/11; C12N 2310/14; C12N 2310/312; C12N 2310/315; C12N 2310/321; C12N 2310/322; C12N 2310/3231; C12N 2320/51

(57) ABSTRACT

Chemical modifications that find use in making oligonucleotide therapeutics and research tools are described.

### CROSS-REFERENCE TO RELATED APPLICATIONS

This application claims priority to U.S. Provisional Patent Application No. 63/274,193, filed Nov. 1, 2021. The entire content of the aforementioned patent application is incorporated herein by reference.

### FIELD OF THE DISCLOSURE

Chemical modifications that find use in making and stabilizing oligonucleotide therapeutics and research tools are described.

### BACKGROUND

Chemical modifications are well-established as being central to the development of oligonucleotides for therapeutics and research uses. The most common and well-known internal oligonucleotide modifications used are phosphorothioate, 2′-OMe and 2′-F. These modifications significantly increase an oligonucleotide's nuclease stability and result in accumulation in target cells, tissues of organs. Their combination, in the context of siRNA, results in significant longevity and potency of knockdown in vitro and in vivo. Additional modifications required for siRNA activity in vivo include attachment of targeting ligands, such as GalNac, hydrophobic moieties and macromolecules.

The prolonged in vivo efficacy of siRNA therapeutics requires additional stabilization of the 5′-phosphate of the guide (antisense) strand of siRNA, which is essential for the productive loading into the RISC complex, RNAi activity and stability.

The only currently available modification used for this purpose, which provides the stability of the terminal phosphate and compatible with RNAi activity, is vinyl-phosphonate.

What is needed are further agents to stabilize the 5′-phosphate of oligonucleotide molecules.

### SUMMARY

Accordingly, the present disclosure relates to synthesis and the use of the different phosphate modifications which can be used for both 5′-phosphate stabilization and internal stabilization in the context of siRNA and other oligonucleotide therapeutics.

In aspects, there is provided a composition comprising an oligonucleotide, the oligonucleotide having complementarity to a target and not being a gapmer or antagomir molecule and comprising one or more stabilized phosphate groups, the stabilized phosphate groups comprising at least one mesyl phosphonate group. In embodiments, the mesyl phosphonate group is a 5′-mesyl phosphonate or internal mesyl phosphonate. In embodiments, the antisense agent is selected from a double-stranded siRNA, single-stranded RNA, and a microRNA.

In aspects, there is provided a method of treating or preventing a disease or disorder comprising administering to a subject in need thereof a therapeutically effective amount of the pharmaceutical composition described herein.

In aspects, there is provided a method of gene silencing comprising contacting a cell comprising the gene with a composition described herein.

In aspects, there is provided a method of making an oligonucleotide, the oligonucleotide comprising at least one mesyl phosphonate (e.g. a 5′-mesyl phosphonate or internal mesyl phosphonate), as shown in FIG. 4 and/or FIG. 5 and/or Example 1.

In aspects, there is provided a composition comprising an oligonucleotide, the oligonucleotide having complementarity to a target and not being a gapmer molecule and comprising one or more stabilized phosphate groups, the stabilized phosphate groups comprising at least one mesyl phosphonate group, wherein: the mesyl phosphonate group is a 5′-mesyl phosphonate; the oligonucleotide is an antisense agent; and all of the nucleotides of the antisense agent are modified. In embodiments, the nucleotides are modified with one or more of 2′-methoxy, 2′-fluoro modifications, locked nucleic acid (LNA), and phosphorothioate linkages. In embodiments, the composition comprises a fully modified siRNA molecule (chemically modified at 100% of nucleotides) and a mesyl phosphonate at the 5′ end of the guide strand.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows the structure of internal and terminal mesyl-phosphonate modification.

FIGS. 2A-B shows the structures of single-stranded oligonucleotides, containing one or more mesyl-phosphonate modifications

FIGS. 3A-B shows the structure of siRNA duplexes containing 5′-P mesyl-phosphonate with or without additional internal mesyl-phosphonate modifications. FIG. 3A shows a duplex where a plurality of hydroxyls at 2′ positions of ribose residues are modified (2′-OMe, 2′-F, 2′-H), and contains any number of Ps (Duplex length from 12 to 24 bp, Antisense strand from 18 to 24 bases, Sense strand from 12 to 26 bases). FIG. 3B shows a duplex where all of hydroxyls at 2′ positions of ribose residues are modified (2′-OMe, 2′-F, 2′-H), and contains any number of Ps (Duplex length from 12 to 24 bp, Antisense strand from 18 to 24 bases, and Sense strand from 12 to 26 bases).

FIG. 4 shows a scheme of synthesis of the internal mesyl-phosphonate modification of the disclosure.

FIG. 5 shows a scheme of the mesyl-phosphonate modification of the disclosure.

FIG. 6 shows that siRNAs with 5′- and internal mesyl-phosphonate modifications demonstrate siRNA activity.

### DETAILED DESCRIPTION

The present disclosure demonstrates, inter alia, that incorporation of a mesyl-phosphonate modification in multiple internal positions of double-stranded siRNA and at the 5′-position of the antisense (guide) strand of chemically modified siRNAs is compatible with their biological functionality. The use of these modifications, e.g. in the context of chemically modified siRNAs (e.g. one or more of 2′-F, 2′-OMe and Ps modifications), increases in vivo stability and longevity of biological effect in vitro and in vivo.

To date, the use of the internal mesyl-phosphonate modification been demonstrated activity in the context of antisense gapmer and antagomir oligonucleotides, where their introduction in multiple positions results in increased stability and efficacy of ASOs. (PNAS Jan. 22, 2019 116 (4) 1229-1234, DOI: 10.1073/pnas. 1813376116; PNAS Dec. 22, 2020 117 (51) 32370-32379; DOI: 10.1073/pnas.2016158117, Nucleic Acids Res. 2021 Sep. 20; 49(16):9026-9041. doi: 10.1093/nar/gkab718).

In aspects, there is provided a composition comprising an oligonucleotide, the oligonucleotide having complementarity to a target and not being a gapmer molecule and comprising one or more stabilized phosphate groups, the stabilized phosphate groups comprising at least one mesyl phosphonate group, e.g. where the mesyl phosphonate group is a 5′-mesyl phosphonate or internal mesyl phosphonate.

In aspects, there is provided a composition comprising an oligonucleotide, the oligonucleotide having complementarity to a target and not being a gapmer molecule and comprising one or more stabilized phosphate groups, the stabilized phosphate groups comprising at least one mesyl phosphonate group, wherein: the mesyl phosphonate group is a 5′-mesyl phosphonate; the oligonucleotide is an antisense agent; and all of the nucleotides of the antisense agent are modified. In embodiments, the nucleotides are modified with one or more of 2′-methoxy, 2′-fluoro modifications, locked nucleic acid (LNA), and phosphorothioate linkages. In embodiments, the composition comprises a fully modified siRNA molecule (chemically modified at 100% of nucleotides) and a mesyl phosphonate at the 5′ end of the guide strand.

In embodiments, the oligonucleotide demonstrates improved nuclease stability as compared to a comparable oligonucleotide without a mesyl phosphonate group (e.g. 5′-mesyl phosphonate or internal mesyl phosphonate). In embodiments, the oligonucleotide demonstrates prolonged in vivo efficacy as compared to a comparable oligonucleotide without a mesyl phosphonate group (e.g. 5′-mesyl phosphonate or internal mesyl phosphonate). In embodiments, the oligonucleotide demonstrates improved loading into the RISC complex, as compared to a comparable oligonucleotide without a mesyl phosphonate group (e.g. 5′-mesyl phosphonate or internal mesyl phosphonate)

In embodiments, the oligonucleotide is an antisense agent. In embodiments, the antisense agent functions through RISC or Ago2. In embodiments, the antisense agent is selected from a double-stranded siRNA, single-stranded RNA, and a microRNA. In embodiments, the antisense agent is a double-stranded siRNA and comprises the mesyl phosphonate group (e.g. 5′-mesyl phosphonate or internal mesyl phosphonate) on the guide (antisense) strand. In embodiments, the antisense agent is a double-stranded siRNA and comprises the mesyl phosphonate group (e.g. 5′-mesyl phosphonate or internal mesyl phosphonate) on the passenger strand.

Interfering nucleic acids generally include a sequence of cyclic subunits, each bearing a base-pairing moiety, linked by intersubunit linkages that allow the base-pairing moieties to hybridize to a target sequence in a nucleic acid (typically RNA) by Watson-Crick base pairing, to form a nucleic acid: oligomer heteroduplex within the target sequence. Interfering RNA molecules include, but are not limited to, antisense molecules, siRNA molecules, single-stranded siRNA molecules, miRNA molecules and shRNA molecules. Such an interfering nucleic acids can be designed to block or inhibit translation of mRNA or to inhibit natural pre-mRNA splice processing, or induce degradation of targeted mRNAs, and may be said to be directed to or targeted against a target sequence with which it hybridizes. Interfering nucleic acids may include, for example, peptide nucleic acids (PNAs), locked nucleic acids (LNAs), 2′-fluoro, 2′-O-Methyl oligonucleotides and RNA interference agents (siRNA agents). RNAi molecules generally act by forming a heteroduplex with the target molecule, which is selectively degraded or knocked down, hence inactivating the target RNA. Under some conditions, an interfering RNA molecule can also inactivate a target transcript by repressing transcript translation and/or inhibiting transcription of the transcript. An interfering nucleic acid is more generally said to be targeted against a biologically relevant target, such as a protein, when it is targeted against the nucleic acid of the target in the manner described above.

In embodiments, an oligonucleotide specifically hybridizes to a target polynucleotide if the oligonucleotide hybridizes to the target under physiological conditions, with a Tm substantially greater than about 45° C., or at least about 50° C., or at least about 60° C. to about 80° C. or higher. Such hybridization corresponds to stringent hybridization conditions. At a given ionic strength and pH, the Tm is the temperature at which about 50% of a target sequence hybridizes to a complementary polynucleotide. Such hybridization may occur with near or substantial complementarity of the antisense oligomer to the target sequence, as well as with exact complementarity.

In embodiments, oligonucleotide comprises at least one strand having a length of about 12 to about 28 nucleotides, or about 14 to about 25 nucleotides, or about 16 to about 20 nucleotides.

In embodiments, the oligonucleotide comprises at least one strand having at least about 16 contiguous nucleotides.

In embodiments, the antisense strand of an siRNA described herein is at least 19 nucleotides (nt) in length. In some embodiments, the antisense strand of an siRNA described herein is 19 to 21 nt in length (i.e., 19, 20 or 21 nt in length). In embodiments, at least 13, 14, 15, 16, 17, 18, 19, 20 or 21 nt of the antisense strand are complementary to the target.

In embodiments, the target is or comprises mRNA (messenger RNA), microRNA, piRNA (piwi-interacting RNA), a coding DNA sequence or a noncoding DNA sequence.

In embodiments, the target is or comprises mammalian mRNA or viral mRNA. In embodiments, the target is an intronic region of the mRNA.

In embodiments, the oligonucleotide comprises one or more of internal mesyl-phosphonate modifications. In embodiments, the oligonucleotide comprises a mesyl-phosphonate modification at the 5′-phosphate. In embodiments, the oligonucleotide comprises one or more of internal mesyl-phosphonate modifications and a mesyl-phosphonate modification at the 5′-phosphate.

In embodiments, the oligonucleotide provided herein comprises a further chemical modification. In embodiments, the modification facilitates the penetration of a cellular membrane in the absence of a delivery vehicle.

In embodiments, the oligonucleotide provided herein comprises at least one nucleotide included in the nucleic acid molecule which is substituted with at least one selected from a hydrogen atom, a fluorine atom, an —O-alkyl group, an —O-acyl group and an amino group. In embodiments, the oligonucleotide provided herein comprises at least one nucleotide included in the nucleic acid molecule which is substituted with an O-methyl group.

In embodiments, the modification is a 2′-O-methylated nucleoside, 2′-fluoro nucleoside, a phosphorothioate bond. a ligand for a cellular receptor, e.g. N-acetylgalactosamine, or a hydrophobic moiety. In some embodiments, the chemical modification is a hydrophobic moiety.

In some embodiments, the hydrophobic moiety is a cholesterol moiety.

In embodiments, the oligonucleotide described herein is devoid of vinyl-phosphonate modifications.

In embodiments, the oligonucleotide described herein can employ a variety of oligonucleotide chemistries. Examples of oligonucleotide chemistries include, without limitation, peptide nucleic acid (PNA), linked nucleic acid (LNA), phosphorothioate, 2′O-Me-modified oligonucleotides, and morpholino chemistries, including combinations of any of the foregoing. Phosphorothioate and 2′-O-Me- and 2′-F modified chemistries are often combined to generate 2′-O-Me/2′-F-modified oligonucleotides having a phosphorothioate backbone. See, e.g., PCT Publication Nos. WO/2013/112053 and WO/2009/008725, each of which is hereby incorporated by reference in its entirety.

In embodiments, the oligonucleotide comprises at least one further modification selected from 2′-methoxy, 2′-fluoro modifications, and LNA.

In embodiments, the phosphate backbone of at least one nucleotide in the oligonucleotide is substituted with phosphorothioate.

In embodiments, the oligonucleotide comprises at least one of each 2′-methoxy, 2′-fluoro, LNA, and phosphorothioate modifications.

2′-O-methylated nucleosides carry a methyl group at the 2′-OH residue of the ribose molecule. 2′-O-Me-RNAs show the same (or similar) behavior as RNA, but are protected against nuclease degradation. 2′-O-Me-RNAs can also be combined with phosphothioate oligonucleotides (PTOs) for further stabilization. 2′-O-Me-RNAs (phosphodiester or phosphothioate) can be synthesized according to routine techniques in the art (see, e.g., Yoo et al., Nucleic Acids Res. 32:2008-16, 2004, which is hereby incorporated by reference).

In embodiments, the 2′-O-methyl nucleoside is positioned at the 3′ terminus of the sense strand. In some embodiments, 3′ terminal region of the sense strand comprises a plurality of 2′-O-methylated nucleosides (e.g., 2, 3, 4, 5 or 6 2′-O-methylated nucleosides within 6 nucleosides of the 3′ terminus). In some embodiments, the 2′-O-methyl nucleoside is positioned at the 3′ terminus of the antisense strand. In some embodiments, 3′ terminal region of the antisense strand comprises a plurality of 2′-O-methylated nucleosides (e.g., 2, 3, 4, 5 or 6 2′-O-methylated nucleosides within 6 nucleosides of the 3′ terminus). In some embodiments, both the 3′ terminal region of the sense strand and the 3′ terminal region of the antisense strand comprise a plurality of 2′-O-methylated nucleosides. In some embodiments, the sense strand comprises 2′-O-methylated nucleosides that alternate with unmodified nucleosides. In some embodiments, the sense strand comprises a contiguous sequence of 2, 3, 4, 5, 6, 7 or 8 2′-O-methylated nucleosides that alternate with unmodified nucleosides. In some embodiments, the anti-sense strand comprises 2′-O-methylated nucleosides that alternate with unmodified nucleosides. In some embodiments, the anti-sense strand comprises a contiguous sequence of 2, 3, 4, 5, 6, 7 or 8 2′-O-methylated nucleosides that alternate with unmodified nucleosides.

In embodiments, the 2′-F nucleoside is positioned at the 3′ terminus of the sense strand. In some embodiments, 3′ terminal region of the sense strand comprises a plurality of 2′-F nucleosides (e.g., 2, 3, 4, 5 or 6 2′-F nucleosides within 6 nucleosides of the 3′ terminus). In some embodiments, the 2′-F nucleoside is positioned at the 3′ terminus of the antisense strand. In some embodiments, 3′ terminal region of the antisense strand comprises a plurality of 2′-F nucleosides (e.g., 2, 3, 4, 5 or 6 2′-F nucleosides within 6 nucleosides of the 3′ terminus). In some embodiments, both the 3′ terminal region of the sense strand and the 3′ terminal region of the antisense strand comprise a plurality of 2′-F nucleosides. In some embodiments, the sense strand comprises 2′-F nucleosides that alternate with unmodified nucleosides. In some embodiments, the sense strand comprises a contiguous sequence of 2, 3, 4, 5, 6, 7 or 8 2′-F nucleosides that alternate with unmodified nucleosides. In some embodiments, the anti-sense strand comprises 2′-F nucleosides that alternate with unmodified nucleosides. In some embodiments, the anti-sense strand comprises a contiguous sequence of 2, 3, 4, 5, 6, 7 or 8 2′-F nucleosides that alternate with unmodified nucleosides.

In embodiments, the RNA complex comprises a phosphorothioate bond. Phosphorothioates (or S-oligos) are a variant of normal DNA in which one of the non-bridging oxygens is replaced by a sulfur. The sulfurization of the internucleotide bond reduces the action of endo- and exonucleases including 5′ to 3′ and 3′ to 5′ DNA POL 1 exonuclease, nucleases S1 and P1, RNases, serum nucleases and snake venom phosphodiesterase. Phosphorothioates are made by two principal routes: by the action of a solution of elemental sulfur in carbon disulfide on a hydrogen phosphonate, or by the method of sulfurizing phosphite triesters with either tetraethylthiuram disulfide (TETD) or 3H-1,2-benzodithiol-3-one 1,1-dioxide (BDTD) (see, e.g., Iyer et al., J. Org. Chem. 55, 4693-4699, 1990) or DDTT (Guzaev, Tetrahedron Letters 52, 434-437, 2011). The latter methods avoid the problem of elemental sulfur's insolubility in most organic solvents and the toxicity of carbon disulfide. The TETD, BDTD, and DDTT methods also yield higher purity phosphorothioates.

In embodiments, there is provided a oligonucleotide of two strands, having a duplex length of about 12 to about 24 bp, and/or an antisense strand of about 18 to about 24 bases, and/or sense strand of about 12 to about 26 bases.

In embodiments, there is provided an oligonucleotide in which all of hydroxyls at 2′ positions of ribose residues are modified (e.g. with one or more of 2′-OMe, 2′-F, and 2′-H), and comprises any number of Ps linkages.

In embodiments, there is provided a oligonucleotide of two strands, having a duplex length of about 12 to about 24 bp, and/or an antisense strand of about 18 to about 24 bases, and/or sense strand of about 12 to about 26 bases; and all of hydroxyls at 2′ positions of ribose residues are modified (e.g. with one or more of 2′-OMe, 2′-F, and 2′-H), and comprises any number of Ps linkages.

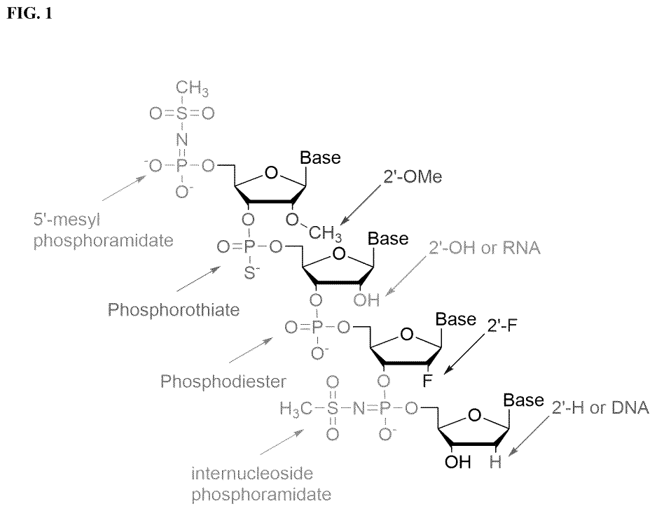

In embodiments, there is provided a composition of the structure of, or substantially of, FIG. 1.

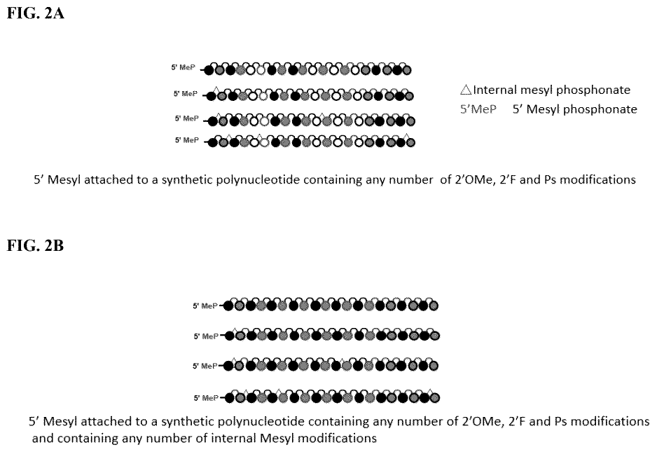

In embodiments, there is provided a composition of the structure of, or substantially of, FIGS. 2A-B.

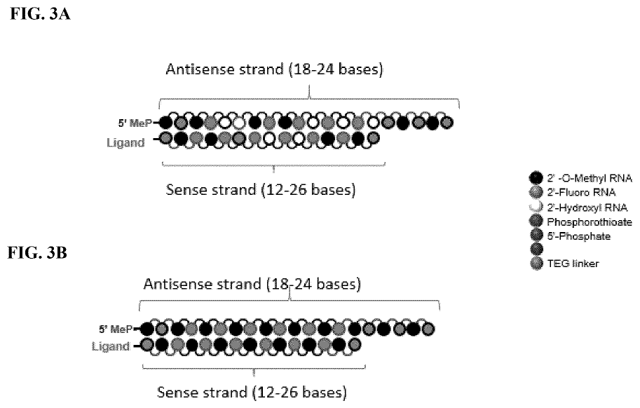

In embodiments, there is provided a composition of the structure of, or substantially of, FIGS. 3A-B.

In embodiments, there is provided a pharmaceutical composition comprising the composition described herein and a pharmaceutically acceptable carrier. A pharmaceutically-acceptable carrier, in embodiments, is a pharmaceutically-acceptable material, composition or vehicle, such as a liquid or solid filler, diluent, excipient, or solvent encapsulating material. A pharmaceutically-acceptable carrier, in embodiments, may include lactose, dextrose, sucrose, sorbitol, mannitol, xylitol, erythritol, maltitol, starch, acacia rubber, alginate, gelatin, calcium phosphate, calcium silicate, cellulose, methyl cellulose, polyvinylpyrrolidone, water, methylhydroxybenzoate, propylhydroxybenzoate, magnesium stearate and mineral oils.

In embodiments, the composition described herein is complexed with or associated with a lipid, the lipid optionally being selected from cholesterol, tocopherol, and a long-chain fatty acid having 10 or more carbon atoms.

In embodiments, the composition described herein is complexed with or associated with N-acetylgalactosamine (GalNac).

The pharmaceutical composition may additionally contain fillers, anti-aggregating agents, lubricants, wetting agents, perfumes, emulsifiers and preservatives. Also, the pharmaceutical composition of the present invention may be formulated using a method well known in the art, such that it can provide the rapid, sustained or delayed release of the active ingredient after administration to mammals. The formulation may be in the form of sterile injection solutions, etc.

In aspects, there is provided a method of treating or preventing a disease or disorder comprising administering to a subject in need thereof a therapeutically effective amount of the pharmaceutical composition described herein.

In aspects, there is provided a method of gene silencing comprising contacting a cell comprising the gene with a composition described herein.

In embodiments, a subject is a human or non-human animal selected for treatment or therapy.

In embodiments, a therapeutically-effective amount is the amount of an agent which is effective for producing the desired therapeutic effect in at least a sub-population of cells in a subject at a reasonable benefit/risk ratio applicable to any medical treatment.

In embodiments, treating a disease in a subject or treating a subject having a disease refers to subjecting the subject to a pharmaceutical treatment, e.g., the administration of a drug, such that at least one symptom of the disease is decreased or prevented from worsening.

In embodiments, an agent that prevent a disorder or condition refers to a agent that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

In embodiments, the pharmaceutical compositions disclosed herein may be delivered by any suitable route of administration, including topically, through inhalation, orally, and parenterally. In certain embodiments, the pharmaceutical compositions are delivered systemically (e.g., via oral or parenteral administration). In certain other embodiments, the pharmaceutical compositions are delivered locally through inhalation into the lungs or topically onto the skin. In some embodiments, the pharmaceutical composition is administered via intradermal injection.

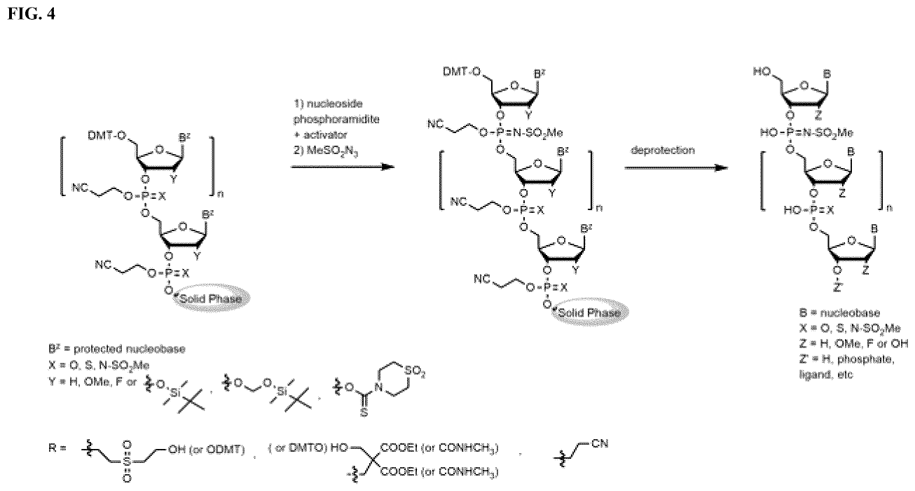

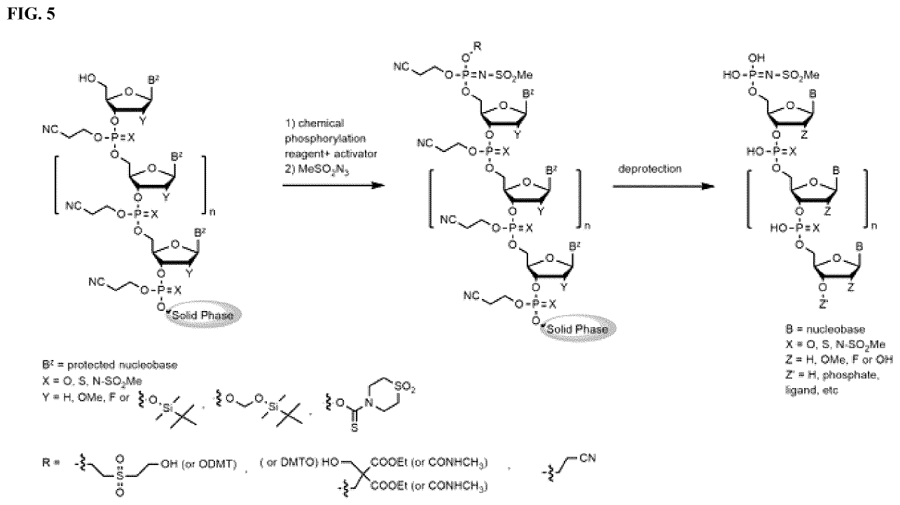

In aspects, there is provided a method of making an oligonucleotide, the oligonucleotide comprising at least one mesyl phosphonate (e.g. 5′-mesyl phosphonate or internal mesyl phosphonate), as shown in FIG. 4 and/or FIG. 5 and/or Example 1. Minor deviations to from the methods of FIG. 4 and/or FIG. 5 and/or Example 1 are also encompassed by the present aspects.

This disclosure is further illustrated by the following non-limiting examples.

### EXAMPLES

### Example 1: Oligonucleotides Synthesis

Oligonucleotide were synthesized as shown in FIG. 4 and FIG. 5.

Oligonucleotides were synthesized by using phosphoramidite solid-phase method using MM-192 (Bioautomation) DNA/RNA oligonucleotide synthesizer. Protected dT, 2′-O-TBDMS- and 2′-OMe ribonucleoside 3′-phosphoramidites, dT or Unylinker-CPG (500 Å) and S-ethylthio-1H-tetrazole were purchased from ChemGenes. Chemical Phosphorylation Reagents: 2-[2-(4,4′-Dimethoxytrityloxy)ethylsulfonyl]ethyl-(2-cyanoethyl)-(N,N-diisopropyl)-phosphoramidite, [3-(4,4′-Dimethoxytrityloxy)-2,2-dicarboxyethyl]propyl-(2-cyanoethyl)-(N,N-diisopropyl)-phosphoramidite, Bis-cyanoethyl-N,N-diisopropyl CED phosphoramidite were purchased from Glen Research and ChemGenes.

Internal mesyl phosphoramidate linkage and 5′-modification were installed by coupling of a protected 2′-deoxy or 2′-OMe nucleoside phosphoramidite or chemical phosphorylation reagent followed by a reaction with methanesulfonyl azide (0.5 M in acetonitrile, 30 min at ambient temperature) instead of iodine oxidation step.

Oligonucleotides were deprotected using AMA-1:1 (v/v) conc. aq. ammonia and 40% aq. methylamine at 65° C. for 1.5 h followed by 2′-deprotection (Et3N*3HF, 55° C., 3 h). HPLC analysis and purification of oligonucleotides were carried out using GE Healthcare HPLC system equipped with an autosampler and a fraction collector.

Ion exchange chromatography was used to purify modified oligonucleotides: 7.5×75 mm TSK-gel SuperQ-5 PW (10 μm, TOSOH), buffer A—20 mM Tris-HCl (pH 7.0) in 10% acetonitrile, buffer B—20 mM Tris-HCl (pH 7.0), 800 mM sodium perchlorate in 10% acetonitrile; a gradient of buffer B: 0% (1 CV), 0-80% (10 CV); a flow rate of 1 mL/min; temperature 45° C.

ESI-MS analysis for the oligonucleotides was performed using Thermo Scientific LTQ Fleet with Agilent 1200 HPLC system. The HPLC instrument was equipped with the 2.1×50 mm Jupiter C18 column (5 μm, Phenomenex); buffer A: 10 mM diisopropylamine, 15 mM 1,1,1,3,3,3-hexafluoroisopropanol; buffer B: 10 mM diisopropylamine, 15 mM 1,1,1,3,3,3-hexafluoroisopropanol, 80% MeCN. Salts were washed out with buffer A (4 CV) followed by a step of 100% buffer B (2 CV) with a flow rate of 0.3 mL/min; temperature 45° C. The MS analysis of the oligonucleotides was carried out in negative mode (capillary voltage 3500 V, dry temp 160° C.), and raw spectra were deconvoluted using ProMass software (ENovatia, USA).

### Example 2: Activity of Oligonucleotides

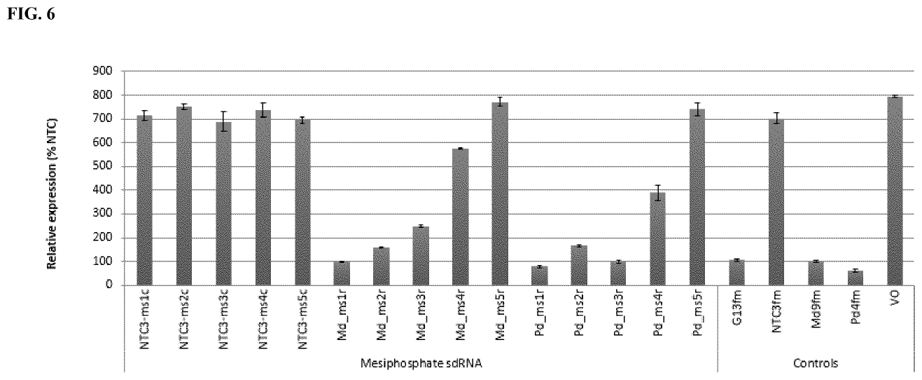

Multiple siRNAs targeting PPIB and Map4K4 as well non-targeting control (NTC) were synthesized as described in FIG. 4 and FIG. 5. siRNA duplexes were formed. HeLa cells were transfected with a dual luciferase reporter plasmid and siRNAs were transfected for 24 hours at 1 uM concentration. Activity was measured essentially as described in Shmushkovich et al (Nucleic Acids Res. 2018 Nov. 16; 46(20):10905-10916) and is shown in FIG. 6.

The following table shows the sequences of the oligonucleotides used in the experiments described in this Example:

| | |
|---|---|
| MAPK4K_1s | phosphate-u(Cf)a(Cf)g(Af)u(Gf)g(Af)a(Uf)us(Uf)sgs(Cf)sus(Gf)su |
| MAPK4K_2s | mesiphosphate-u(Cf)a(Cf)g(Af)u(Gf)g(Af)a(Uf)us(Uf)sgs(Cf)sus(Gf)su |
| MAPK4K_3s | phosphate-u(Cf)a(Cf)g(Af)u(Gf)g(Af)a(Uf)um(Uf)mgm(Cf)mum(Gf)mu |
| MAPK4K_4s | mesiphosphate- |
| | um(Cf)ma(Cf)g(Af)u(Gf)g(Af)a(Uf)us(Uf)sgs(Cf)sus(Gf)su |
| MAPK4K_5s | mesiphosphate- |
| | um(Cf)ma(Cf)g(Af)u(Gf)g(Af)a(Uf)um(Uf)mgm(Cf)mum(Gf)mu |
| PPIB_1s | phosphate-u(Af)g(Af)c(Uf)u(Cf)c(Af)c(Af)gs(Af)sas(Cf)sus(Cf)su |
| PPIB_2s | mesiphosphate-u(Af)g(Af)c(Uf)u(Cf)c(Af)c(Af)gs(Af)sas(Cf)sus(Cf)su |
| PPIB_3s | phosphate-u(Af)g(Af)c(Uf)u(Cf)c(Af)c(Af)gm(Af)mam(Cf)mum(Cf)mu |
| PPIB_4s | mesiphosphate- |
| | um(Af)mg(Af)c(Uf)u(Cf)c(Af)c(Af)gs(Af)sas(Cf)sus(Cf)su |
| PPIB_5s | mesiphosphate- |
| | um(Af)mg(Af)c(Uf)u(Cf)c(Af)c(Af)gm(Af)mam(Cf)mum(Cf)mu |
| NTC3_1s | phosphate-u(Af)g(Gf)a(Af)a(Af)c(Af)u(Gf)u(Af)sas(Af)scs(Cf)sas(Af) |
| NTC3_2s | mesiphosphate- |
| | u(Af)g(Gf)a(Af)a(Af)c(Af)u(Gf)u(Af)sas(Af)scs(Cf)sas(Af) |
| NTC3_3s | phosphate- |
| | u(Af)g(Gf)a(Af)a(Af)c(Af)u(Gf)u(Af)mam(Af)mcm(Cf)mam(Af) |
| NTC3_4s | mesiphosphate- |
| | um(Af)mg(Gf)a(Af)a(Af)c(Af)u(Gf)u(Af)sas(Af)scs(Cf)sas(Af) |
| NTC3_5s | mesiphosphate- |
| | um(Af)mg(Gf)a(Af)a(Af)c(Af)u(Gf)u(Af)mam(Af)mcm(Cf)mam(Af) |

The present disclosure demonstrates synthesis and biological activity of the chemically synthesized siRNAs containing 5′-P and internal mesyl-phosphonate modifications. The use of these modifications enhances stability of siRNAs has utility as a therapeutic agent.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising,” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein.

All publications, patents, and patent publications cited are incorporated by reference herein in their entirety for all purposes.

All of the features disclosed herein may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

What is claimed is:

1. A composition comprising an oligonucleotide, the oligonucleotide having complementarity to a target and not being a gapmer molecule and comprising one or more stabilized phosphate groups, the stabilized phosphate groups comprising at least one mesyl phosphonate group.

2. The composition of claim 1, wherein the mesyl phosphonate group is a 5′-mesyl phosphonate or an internal mesyl phosphonate.

3. The composition of claim 1 or 2, wherein the oligonucleotide demonstrates improved nuclease stability as compared to a comparable oligonucleotide without a mesyl phosphonate group and/or demonstrates prolonged in vivo efficacy as compared to a comparable oligonucleotide without a mesyl phosphonate group.

4. The composition of any one of claims 1-3, wherein the oligonucleotide is an antisense agent.

5. The composition of claim 4, wherein the antisense agent functions through RISC or Ago2.

6. The composition of any one of claims 4-5, wherein the antisense agent is selected from a double-stranded siRNA, single-stranded RNA, and a microRNA.

7. The composition of any one of claims 4-6, wherein the antisense agent is a double-stranded siRNA and comprises the mesyl phosphonate group on the guide (antisense) strand and, optionally, wherein the mesyl phosphonate group is a 5′-mesyl phosphonate group.

8. The composition of any one of claims 4-6, wherein the antisense agent is a double-stranded siRNA and comprises the mesyl phosphonate group on the passenger strand and, optionally, wherein the mesyl phosphonate group is a 5′-mesyl phosphonate group.

9. The composition of any one of claims 1-7, wherein the oligonucleotide comprises at least one strand having a length of about 12 to about 28 nucleotides, or about 14 to about 25 nucleotides, or about 16 to about 20 nucleotides.

10. The composition of any one of claims 1-7, wherein the oligonucleotide comprises at least one strand having a length of about 12 to about 28 nucleotides, or about 14 to about 25 nucleotides, or about 16 to about 20 nucleotides.

11. The composition of any one of claims 1-7, wherein the oligonucleotide comprises at least one strand having at least about 16 contiguous nucleotides.

12. The composition of any one of claims 1-11, wherein the target is or comprises mammalian mRNA or viral mRNA.

13. The composition of claim 12, wherein the target is an intronic region of the mRNA.

14. The composition of any one of claims 1-13, wherein the oligonucleotide comprises one or more of internal mesyl-phosphonate modifications.

15. The composition of any one of claims 1-14, wherein the oligonucleotide comprises at least one further modification selected from 2′-methoxy, 2′-fluoro modifications, and locked nucleic acid (LNA).

16. The composition of any one of claims 1-15, wherein the oligonucleotide comprises at least one phosphorothioate linkages.

17. The composition of any one of claims 1-16, wherein the oligonucleotide comprises at least one of each 2′-methoxy, 2′-fluoro, LNA, and phosphorothioate modifications.

18. A pharmaceutical composition comprising the composition of any one of claims 1-17 and a pharmaceutically acceptable carrier.

19. A method of treating or preventing a disease or disorder comprising administering to a subject in need thereof a therapeutically effective amount of a composition of any one of claims 1-17 or the pharmaceutical composition of claim 18.

20. A method of gene silencing comprising contacting a cell comprising the gene with a composition of any one of claims 1-17 or the pharmaceutical composition of claim 18.

21. A method of making an oligonucleotide, the oligonucleotide comprising at least one mesyl phosphonate, as shown in FIG. 4 and/or FIG. 5 and/or Example 1.

22. A composition comprising an oligonucleotide, the oligonucleotide having complementarity to a target and not being a gapmer molecule and comprising one or more stabilized phosphate groups, the stabilized phosphate groups comprising at least one mesyl phosphonate group, wherein:
the mesyl phosphonate group is a 5′-mesyl phosphonate;
the oligonucleotide is an antisense agent; and
all of the nucleotides of the antisense agent are modified.

23. The composition of claim 22, wherein the nucleotides are modified with one or more of 2′-methoxy, 2′-fluoro modifications, locked nucleic acid (LNA), and phosphorothioate linkages.
